(19) United States
(12) Patent Application Publication
(10) Pub. No.: US 2016/0243323 A1
(43) Pub. Date: Aug. 25, 2016

(54) Barrier Device

(71) Applicant: Kevin W. Burns, New Brunswick, NJ (US)

(72) Inventor: Kevin W. Burns, New Brunswick, NJ (US)

(21) Appl. No.: 14/629,789

(22) Filed: Feb. 24, 2015

(51) Int. Cl. A61M 16/00; B32B 37/12; A61M 16/20

(52) U.S. Cl. CPC A61M 16/0048; A61M 16/208; B32B 37/12; B32B 2327/06; B32B 2331/04; B32B 2535/00

(57) ABSTRACT

A barrier device used to perform ventilations on a victim undergoing cardiac arrest may be formed having similar dimensions as a standard identification card. Such a barrier device may also include identification information imprinted on a rear side, as well as instructions for how to perform CPR on a front side. A one-way valve is provided in the combined barrier device and identification card.

### FIELD

The present application relates generally to the field of barrier devices used to administer cardio-pulmonary resuscitation (CPR). More particularly, the present application relates to an improved personal barrier device (e.g., personal barrier, breathing barrier, CPR device, resuscitation device, etc.) that may be conveniently carried for personal use. The present application also relates to an improved, combined barrier device and certification card for CPR. The present application also relates to an improved method for manufacturing a combined barrier device and certification card.

### BACKGROUND

Cardiac arrest (e.g., pulselessness, clinical death, etc.) refers to a medical emergency characterized by the failure of a person's heart to contract effectively, such that the victim's blood stops circulating. CPR refers to an emergency medical procedure used to provide artificial circulation of oxygenated blood to a person undergoing cardiac arrest. In particular, CPR is comprised of a sequence (e.g., a pattern, series, etc.) of chest compressions and mouth ventilations (e.g., breaths, ventilations, etc.). A recommended ratio of chest compressions to ventilations may vary from person to person based on various factors (e.g., age). For instance, a ratio of 30 compressions to 2 ventilations may be recommended for adults, whereas a ratio of 15 compressions to 2 ventilations may be recommended for children.

People may go into cardiac arrest for many reasons. For example, heart disease may be viewed as the leading cause of cardiac arrest, as well as the leading cause of death in the United States. Cardiac arrest may also be caused, for example, by events such as trauma, excessive bleeding, and drowning.

A person who goes into cardiac arrest, if left untreated, will begin to suffer irreversible brain damage after a few minutes (e.g., approximately 3-4 minutes). Death may follow after approximately five minutes of going into cardiac arrest. According to the American Heart Association (AHA), about 92 percent of sudden cardiac arrest victims die before reaching the hospital, but statistics prove that if more people knew CPR, more lives may be saved. The AHA also reports that immediate CPR can double, or even triple, a victim's chance of survival. Further, in some cases, the act of applying CPR, on its own, may actually be sufficient to restore normal heart operation and blood circulation. Therefore, it is generally advantageous to perform CPR as soon as possible after a person goes into cardiac arrest.

The compressions used to perform CPR may be performed without any special equipment. For example, compressions may be adequately performed by placing a heel of one hand over the center of a person's chest, placing the opposite hand over the first hand, and pushing downward (i.e., into the person's chest) approximately two inches (e.g., approximately 5 cm). In contrast, with regards to ventilations, it may be generally advisable to use a personal barrier device (e.g., a breathing barrier) during CPR. A barrier device may be used for several reasons. For example, a barrier device may be used to prevent direct facial contact between a rescuer (i.e., one who is performing CPR) and a victim undergoing cardiac arrest, to prevent the rescuer from ingesting the victim's bodily fluids, and to reduce the likelihood of disease transmission between the rescuer and victim.

However, people who are trained to perform CPR may choose against carrying a barrier device on their person. For example, people may generally wish to carry a limited amount of “stuff” in their pockets. For instance, a person's decision to carry something in his or her pockets may be based on a cost/value determination in which the space required to carry the object is weighed against the personal value of keeping the object close at hand. In some cases, barrier devices may be bulky. Further, barrier devices may also be costly. Thus, in the case of a barrier device, a person's decision may weigh against on-hand carry.

In view of the foregoing uses of personal barrier devices, if those who are trained to perform CPR decide against on-hand carry of a barrier device, then they may be more hesitant to perform ventilations on someone in cardiac arrest. For example, those who are trained to perform CPR, but who do not have a barrier device handy, may be concerned about the possible dangers of a victim transmitting any diseases he or she may have. Thus, those trained in CPR may decide to perform hands-only CPR (i.e., performing CPR with compressions only), or they may decide against performing CPR at all. Hands-only CPR may not be as effective as conventional CPR (i.e., CPR comprising a sequence of compressions and ventilations) because hands-only CPR may circulate blood that contains less oxygen than conventional CPR.

Accordingly, it would be advantageous to provide a barrier device design that addresses one or more of the issues discussed above, that is relatively simple and efficient to manufacture, and is relatively inexpensive. These and other advantageous features as discussed herein will be apparent to those reviewing the present application.

### SUMMARY

According to an exemplary embodiment, a barrier device includes a card including a top element and a bottom element coupled to a middle element. The middle element is sandwiched between the top and bottom elements. Also, the middle element includes a hole disposed therethrough. The top element includes a hole disposed therethrough, and the hole of the top element is smaller than the hole of the middle element. A flap is cut into the bottom element by cutting one or more lines therethrough, the hole of the top element is generally superimposed over the hole of the middle element when the top element is positioned over the middle element, and the flap and the hole of the top element cooperatively define a one-way valve.

According to another exemplary embodiment, a barrier device includes a card having a length dimension, a width dimension, and a height dimension, the length and width dimensions being substantially greater than the height dimension. The card includes a one-way valve, and the one-way valve is coupled to a hole disposed within the card.

According to yet another exemplary embodiment, a method for manufacturing a barrier device includes the following steps: providing a planar middle element having a hole formed therein, a planar top element having a hole formed therein, and a planar bottom element having a flap formed therein, raising the flap from portions of the bottom element surrounding the flap, and coupling the middle, top, and bottom elements together. The middle element of the barrier device is positioned between the top and bottom elements.

### BRIEF DESCRIPTION OF THE DRAWINGS

FIG. 1 is a top plan view of a barrier device, which illustrates an intake side thereof, according to an exemplary embodiment.

FIG. 2 is a bottom plan view of the barrier device shown in FIG. 1, which illustrates an exhaust side thereof, according to an exemplary embodiment.

FIG. 3 is a side plan view of the barrier device shown in FIG. 1.

FIG. 4 is an exploded view of a top element, a middle element, and a bottom element of a barrier device, according to an exemplary embodiment.

FIG. 5 illustrates a rescuer performing ventilations on a victim in cardiac arrest, while using the barrier device shown in FIG. 1.

### DETAILED DESCRIPTION

Individuals who work in the medical industry (e.g., paramedics, nurses, doctors, etc.), first responders (e.g., firefighters, police officers, etc.), and professionals in other fields may be required to attend a CPR certification class as a condition of their employment. In order to address this need, certifying organizations, such as the AHA and the Red Cross, regularly host classes to certify people in CPR. Upon passing a CPR certification class, a certifying organization may generally issue attendees a certification card. Such CPR certification cards may typically be configured to fit inside wallets, money clips, checkbooks, etc. In other words, a CPR certification card may have a standard size corresponding to other cards (e.g., credit cards, driver's licenses, business cards, etc.) typically carried in wallets, money clips, etc.

As described in the background section above, people may decide against carrying conventional barrier devices, for various reasons. For example, conventional barrier devices may be too bulky and/or too expensive for everyday on-hand carry. Disadvantages such as large size and high cost do not generally apply to standard-sized cards, such as a driver's license or credit card. For example, standard-sized cards are practical for everyday carry because they are relatively thin, and easily fit in wallets and money clips. Standard-sized cards are also relatively simple and inexpensive to manufacture.

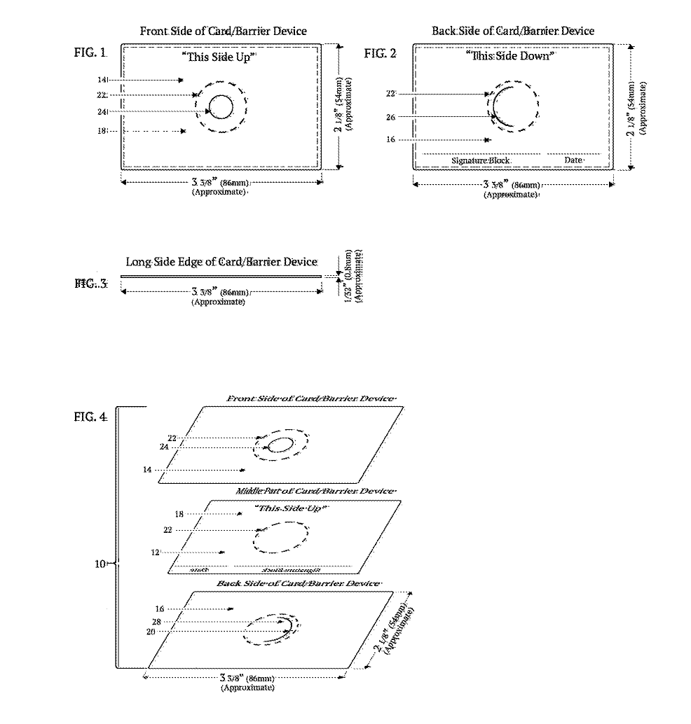

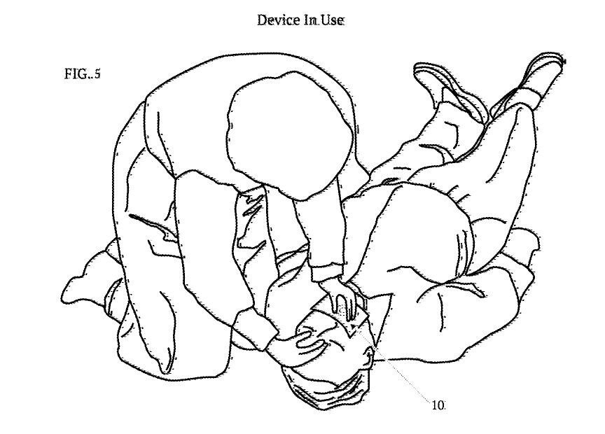

According to an exemplary embodiment, referring to FIGS. 1-3, an improved barrier device 10 is configured to easily fit within a compartment, such as a sleeve in a wallet, card case, or checkbook, or within a money clip. For example, according to an exemplary embodiment, the overall dimensions of the barrier device 10 may generally correspond to the dimensions of a standard driver's license, credit card, or business card. In particular, according to an exemplary embodiment, the dimensions of the barrier device 10 may be approximately 3⅜″ long, 2⅛″ wide, and 1/32″ thick (e.g., approximately 8.57 cm long, 5.4 cm wide, and 0.03 cm thick). According to another exemplary embodiment, the overall dimensions of the barrier device 10 may be approximately 3.5″ long, 2″ wide, and 1/32″ thick (e.g., approximately 8.9 cm long, 5.08 cm wide, and 0.03 cm thick). Although various dimensions of the barrier device 10 have been disclosed herein, it should be understood that the dimensions of the barrier device 10 may vary according to other exemplary embodiments, and that the present disclosure is not intended to be limited to a barrier device having the specific dimensions disclosed herein. In other words, according to other exemplary embodiments, a barrier device may be longer or shorter, wider or narrower, more thin, or more thick relative to the dimensions of the barrier device 10 disclosed herein.

Referring now to FIG. 4, according to an exemplary embodiment, the barrier device 10 may be primarily comprised of at least three, generally planar elements: a middle element 12 (e.g., member, sheet, card, layer, etc.), a top element 14 positioned on a front side of the middle element 12, and a bottom element 16 positioned on a rear side of the middle element 12. As will be described in more detail below, portions of each of the middle, top, and bottom elements 12, 14, 16 may be removed (e.g., holes or other features may be disposed within each element). The areas where the middle, top, and bottom elements 12, 14, 16 overlap may generally define a body 18 of the barrier device 10. In other words, the body 18 is defined by portions of the middle, top, and bottom elements 12, 14, 16 that overlap when the middle, top, and bottom elements 12, 14, 16 are superimposed over each other.

According to an exemplary embodiment, the middle, top, and bottom elements 12, 14, 16 are coupled to one another. For example, the top and bottom elements 14, 16 may be adhesively coupled to the middle element 12 via an adhesive (e.g., glue, paste, double-sided tape, etc.). According to another exemplary embodiment, the top and bottom elements 14, 16 may be thermally bonded (e.g., thermally laminated) to the middle element 12. According to other exemplary embodiments, the top and bottom elements 14, 16 may be coupled to the middle member 12 in any suitable manner, and the methods described herein are not intended to be limiting.

According to an exemplary embodiment, the body 18 of the barrier device 10 may be generally formed from a laminated cardstock. In other words, the middle member 12 may be formed of a paperboard (e.g., cardstock, fiberboard, etc.) which is sandwiched between a pair of transparent, polymeric sheets (e.g., the top and bottom members 14, 16). According to an exemplary embodiment, the middle member 12 is opaque. Due to the opaque nature of the middle member 12, information may be imprinted on both sides thereof. For example, a middle member 12 may include identification information imprinted on a rear side thereof (corresponding to an exhaust side of the barrier device 10), and instructions to perform CPR may be imprinted on a front side thereof (corresponding to an intake side of the barrier device 10). Of course, it should be understood that identification information and instructions to perform CPR may be imprinted on either side of the middle member 12. The polymeric top and bottom sheets may be, for example, thermo laminating sheets, self-adhesive laminating sheets, or any other suitable transparent, polymeric sheets. Thus, when the top and bottom elements 14, 16 are coupled to the middle member 12, identification information and/or instructions for performing CPR may be visible therethrough. Further, according to another exemplary embodiment, the top and bottom elements 14, 16, while generally transparent, may be imprinted with identification information and/or instructions for performing CPR.

According to another exemplary embodiment, a barrier device, such as the barrier device 10, may be formed from a generally opaque polymeric material. For example, polyvinyl chloride (PVC), polyvinyl acetate (PVA), polyvinyl chloride acetate (PVCA), or any other suitable polymeric material may be used to form the barrier device 10. In particular, materials that are used to make standard driver's licenses and credit cards may be used. Due to the opaque nature of the polymeric material used to form such a barrier device 10, information may be imprinted on both sides of the barrier device 10. For example, barrier devices formed according to this exemplary embodiment may include identification information imprinted on an exhaust side thereof, and instructions to perform CPR imprinted on an intake side thereof. Of course, it should be understood that identification information and instructions to perform CPR may be imprinted on either side of the barrier device. Further, a barrier device 10 which is formed from such a polymeric material may not require lamination (i.e., the barrier device may be formed without top and bottom elements, such as the top and bottom elements 14, 16 described above). According to other exemplary embodiments, the barrier device 10 may be formed from a composite material, a metal, or any combination of other suitable materials.

Referring to FIGS. 2 and 4, according an exemplary embodiment, the barrier device 10 is configured as an identification card (e.g., a CPR certification card). For example, the barrier device 10 is configured to function as an identification/certification card, as well as a barrier device used to perform ventilations during CPR. In order for the barrier device 10 to function as an identification card, at least a portion of the barrier device 10 may be opaque. Therefore, identification information imprinted on the barrier device 10 may stand out against an opaque background. According to an exemplary embodiment, an exhaust side of the barrier device 10 includes identification information (e.g., a name, identification number, etc.) of a CPR-certified individual displayed on the body 18, which may validate that the person carrying the barrier device 10 is indeed trained to perform CPR. Although FIG. 2 illustrates identification information as being provided on a rear, exhaust side of the barrier device 10, it should be understood that identification information may be provided on a front, intake side of the barrier device 10, according to another exemplary embodiment.

Identification information may be imprinted on the barrier device 10 in any suitable way. For example, the barrier device 10 may include identification information that is punched therein (similar to how a person's name is displayed on the front of a typical credit card), printed thereon (similar to how a person's identification information is imprinted on a standard driver's license), or written thereon (similar to how a person signs their name on the back of a credit card). Further, identification information may be electronically stored in the barrier device. For example, identification information may be stored in a magnetic strip embedded in the barrier device, on a microchip embedded in the barrier device, or on another storage device, such as flash memory. Although several methods of representing identification information on a barrier device have been disclosed herein, this disclosure is not intended to limit the ways in which identification information may be displayed on a barrier device.

Advantageously, the barrier device 10 may replace conventional CPR certification cards, which are generally conveniently sized for on-hand carry. Thus, if the barrier device 10 is substituted for a conventional CPR certification card, those who are certified or trained to perform CPR, and who also carry the barrier device 10 on-hand, may always have a barrier device readily available to perform CPR. Further, in the event that a CPR-certified individual undergoes cardiac arrest himself, and a third-party bystander responds to the situation by going through the victim's wallet and discovers the barrier device 10, then the bystander may be able to identify the CPR-certified individual and inform first responders of his or her identity. Further, as explained in more detail below, the bystander may be able to use the barrier device 10 to perform CPR on the CPR-certified individual undergoing cardiac arrest.

As indicated above, CPR is a life-saving medical procedure performed when a person undergoes cardiac arrest. Emergency situations, such as when one is going through cardiac arrest, can be incredibly stressful, and stressful situations can cause people to hesitate, forget, and falter. Some CPR devices may include instructions for how to perform CPR. For example, some barrier devices may be stored in protective cases, and instructions for how to perform CPR may also be stored in such protective cases. However, every second counts during emergency situations, such as when someone undergoes cardiac arrest, and priceless seconds may be wasted finding and reading the instructions for how to use a barrier device.

Referring now to FIGS. 1, 2, and 4, according to an exemplary embodiment, the barrier device 10 is configured to display instructions to perform CPR on a front, upper surface thereof (e.g., an intake side of the barrier device 10) when it is positioned over a person's mouth. As described above, at least a portion of the barrier device 10 may be opaque. Therefore, instructions for how to perform CPR imprinted on the barrier device 10 may stand out against an opaque background. As shown in FIGS. 1, 2, and 4, instructions to perform CPR may be clearly represented (e.g., printed) on the front, intake side, and/or the rear, exhaust side of the barrier device 10. Of course, the Figures are intended to be illustrative only, and the disclosure herein is not intended to be limited to any specific instructions for how to perform CPR. For example, instructions may in the form of typed words, images, indentations or protrusions (e.g., braille), or any combination thereof.

Advantageously, the instructions printed on the front side of the barrier device 10 are in clear view of the rescuer when the barrier device 10 is in position over a victim's mouth. Further, the instructions displayed on the intake side may indicate, for example, “this side up,” so that a one-way valve of the barrier device 10 is properly oriented, which will be explained in more detail below. Similarly, the exhaust side of the barrier device 10 may include instructions displayed thereon which indicate, for example, “this side facing victim's mouth,” so that a rescuer may properly orient a one-way valve of the barrier device 10. Thus, if a rescuer hesitates, forgets, or otherwise falters while performing CPR on a victim undergoing cardiac arrest, the barrier device 10 ensures that instructions are conveniently in open view of the rescuer.

According to an exemplary embodiment, as shown in FIG. 5, the barrier device 10 is configured to rest relatively flat (e.g., planar) over the mouth of another person (i.e., when that person's back is resting on the ground). For example, the barrier device 10 may be flexible, yet resilient—similar to the flexibility of a driver's license or credit card, for example. Advantageously, the flexible, yet resilient structure of the barrier device 10 allows it to rest generally flat when it is positioned over a person's mouth so that a rescuer may easily and fully read any instructions printed on the front, intake side thereof. Therefore, there is no need to smooth out or stretch the barrier device 10 in order to read instructions printed thereon. Further, the flexible and resilient structure of the barrier device 10 also allows it to be easily inserted in, and removed from, a wallet, money clip, or any other suitable compartment.

According to an exemplary embodiment, the barrier device 10 includes a one-way valve 20 which may be comprised of portions of the top and bottom elements 14, 16. According to an exemplary embodiment, the one-way valve 20 is configured to allow one-directional air flow therethrough. In particular, the one-way valve 20 is configured to allow air to flow from the intake side, and to the exhaust side. Thus, when the barrier device 10 is oriented over the mouth of a victim undergoing cardiac arrest such that the intake side is facing upwardly (away from the victim), a rescuer may blow air through the one-way valve 20 and into the lungs of the victim. The rescuer would not be able to suck air from the exhaust side of the one-way valve, nor would air or bodily fluids from the victim be able to flow from the exhaust side to the intake side of the barrier device 10. Thus, the one-way valve 20 of the barrier device 10 may be used to prevent direct facial contact between a rescuer (i.e., one who is performing CPR) and a victim undergoing cardiac arrest, to prevent the rescuer from ingesting the victim's bodily fluids, and to reduce the likelihood of disease transmission between the rescuer and victim.

The structure and functionality of the one-way valve 20 may be better understood by describing a method of manufacturing the barrier device 10. According to an exemplary embodiment, a method of manufacturing the barrier device 10 may be comprised of approximately four (4) steps. The first step in the method may be forming a hole 22 in the middle element 12. As shown in FIGS. 1-2, the hole 22 is illustrated using a dashed line, and it is formed (e.g., punched, cut, etc.) in a central portion of the middle element 12. The second step in the method may be forming a hole 24 in the top element 14, which is shown in FIGS. 1 and 5 using a solid line. The holes 22, 24 may be generally circular, or the holes 22, 24 may have any suitable shape (semicircular, polygonal, etc.). According to an exemplary embodiment, as shown in FIG. 1, the holes 22, 24 are generally concentric when the top element 14 is coupled to the middle element 12. In other words, the holes 22, 24 are generally concentric when the top element 14 is superimposed over the middle element 12. Further, the hole 24 of the top element 14 may be smaller than the hole 22 of the middle element. In other words, a perimeter of the hole 22 may extend outwardly past a perimeter of the hole 24 when the holes 22, 24 are superimposed over each other. According to another exemplary embodiment, the holes 24, 22 may be substantially the same size. The third step of the method may be forming an arc 26, shown in FIG. 2, in the bottom element 16 to define a flap 28 (e.g., a hood, fold, segment, etc.), which is shown in FIG. 4. According to an exemplary embodiment, a radius of the arc 26 is greater than a radius of the hole 24, and less than a radius of the hole 22. According to an exemplary embodiment, a center of the arc 26 is generally concentric with the holes 22, 24 when the bottom element 16 is coupled to the middle and top elements 12, 14. According to other exemplary embodiments, a flap having another shape (e.g., a polygonic shape in which one or more sides is not cut, such as a [,], <, >, ̂, or V shape) may be formed in the bottom element 16. It should be understood that the three steps described above may be performed in any order. The fourth and final step in the method may include raising or lifting the flap 28 from portions of the bottom element 16 surrounding the flap 28 (in order to space the flap 28 apart from the surrounding portions of the bottom element), and coupling the middle, top, and bottom elements 12, 14, 16 together in order to form the barrier device 10.

According to an exemplary embodiment, the flap 28 and the hole 24 of the top element 14 cooperatively define the one-way valve 20. For example, the flap 28 of a barrier device 10 may be forced outwardly (relative to the exhaust side of the barrier device 10) when a person blows air through the intake side of the barrier device 10. However, because the hole 24 is smaller than the arc 26, the flap 28 is blocked from moving outwardly relative to the intake side of the barrier device 10. Therefore, the flap 28 and the hole 24 cooperate to allow air to flow through the one-way valve 20 in only one direction.

Although not shown in the FIGURES, according to another exemplary embodiment, instead of forming an arc 26 in the bottom element 16, a cut in the form of an “X,” “H,” “+”, or “*” may be formed within the bottom element 16. Forming such a cut within the bottom element 16 may provide a barrier device with a bilateral moving wafer valve. According to another exemplary embodiment, a one-way valve may be configured like a duckbill. For example, a pair of flaps may be provided such that first ends of each flap are engaged, and second ends of each flap opposite the first ends diverge.

According to an exemplary embodiment, the flap 28 is configured to lie flat against an inner surface of the top element 14 defined between the hole 24 and the arc 26. For example, according to an exemplary embodiment, an angle defined by a length of the arc 26 is less than 180°. Because the arc 26 does not extend past 180°, the opposite sides of the flap 28 tend to hold the flap 28 against the inner surface of the top element 14. Although not shown in the FIGURES, the flap 28 could be formed in other ways in order for it to lie flat against the inner surface of the top element 14. For example, the arc 26 may be cut into the bottom element 16 at an angle so that a small portion of an outer perimeter of the flap is covered by a small portion of the bottom element 16 surrounding the flap 28. The engagement of the small portions of the flap 28 and the surrounding bottom element 16 may tend to resist movement of the flap 28 outwardly. However, the force of air being blown through the intake side of the barrier device 10 may be sufficient to overcome any resistance created by cutting the arc 26 at an angle. According to another embodiment, portions of the flap 28 proximate opposite ends of the arc 26 may be configured to bias the flap 28 in a closed position. For example, stresses may be created proximate opposite ends of the arc 26 in order to bias the flap 28 in a closed position. Such stresses may be created, for example, by heating specific portions of the bottom element 16 to hotter temperatures than the rest of the bottom element 16. Advantageously, a barrier device 10 which comprises the one-way valve 20 described above may be easily placed in, and removed from, a compartment (e.g., a wallet, money clip, etc.) without damaging the one-way valve 20, or getting snagged on the one-way valve 20. That is, the barrier device 10 may be slid translationally between planar surfaces (e.g., of other cards or card pockets) without damaging the one-way valve 20.

As shown in FIGS. 2 and 4, the arc 26, which defines the flap 28, is formed such that the portion of the flap 28 which is coupled to the bottom element 16 (i.e., the “un-cut” portion of the flap) is faced (oriented) towards a lengthwise end (e.g., as the barrier device 10 is depicted in FIGS. 2 and 4, a left/right end) of the barrier device 10. According to another exemplary embodiment, the arc 26 may be formed such that a portion of the flap 28 which is coupled to the bottom element 16 is oriented towards a widthwise end (e.g., as the barrier device 10 is depicted in FIGS. 2 and 4, a top/bottom end) of the barrier device 10. According to another exemplary embodiment, the arc 26 may be formed within the bottom element 16 in any suitable orientation.

According to another exemplary embodiment, a second bottom element (not shown) may be coupled to a bottom surface of the bottom element 16. For example, the second bottom element may include a central hole that is larger than the flap 28. The outer dimensions and thickness of the second bottom element may be substantially similar to the outer dimensions of the middle, top, and bottom elements 12, 14, 16. According to other exemplary embodiments, the second bottom element may have any suitable thickness. When the second bottom element is coupled to the bottom element 16, the flap 28 is positioned below an outer surface of the second bottom element. Thus, a second bottom element may be used to further protect the one-way valve 20 and to facilitate placement and removal of the barrier device 10 in a compartment.

Advantageously, when the barrier device 10 is removed from a compartment for use during CPR, no other steps need to be performed in order to begin providing CPR. That is, upon removal of the barrier device 10 from a compartment, the barrier device 10 is already in a ready-to-use configuration. Thus, one who has to use the barrier device 10 to perform CPR does not have to perform any additional steps in order to provide ventilations. The ready-to-use configuration of the barrier device 10 equates to being able to perform CPR in less time. Thus, the ready-to-use configuration of the barrier device 10 may reduce the number of cells that a victim loses during cardiac arrest, and improve a victim's overall chance of survival.

During CPR, it may be desirable to regulate the amount or volume of air used for ventilations. For example, if rescue breaths are delivered too vigorously (which people may be likely to do in a stressful, emergency situation), a victim's stomach may inflate causing the victim to vomit, a victim's airway could possibly become obstructed, and/or a rescuer could be contaminated with the victim's bodily fluids.

According to an exemplary embodiment, the one-way valve 20 is configured to regulate the amount of air that may be blown therethrough. For example, the size of the hole 24, the radius of the arc 26, the degree to which the arc 26 is cut, and the thickness of the flap 28 may be some variables used to determine the flow rate of air through the one-way valve 20.

According to another exemplary embodiment, any suitable one-way valve may be coupled within a card that is configured for everyday carry. For example, a silicone or rubber one-way valve may be coupled in any suitable way to a card, such as an identification card. Additionally, a card used for a barrier device may have any suitable thickness, and be configured in a variety of ways. For example, the barrier devices disclosed herein may include rounded corners, chamfered corners, or square corners.

Although the exemplary embodiments for a barrier device 10 described herein may be inserted, by itself, and removed from compartments such as a sleeve in a wallet or checkbook, or a money clip, a barrier device 10 may also be provided with a protective sleeve, such as the faraday sleeves that usually come with identification cards in which identification information is electronically stored thereon. Such a sleeve may provide additional protection for the barrier device 10 as it is inserted, stored, and/or removed from a compartment such as a sleeve in a wallet or checkbook, or a money clip, purse, or handbag.

As utilized herein, the terms “approximately,” “about,” “substantially,” “essentially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” etc.) are merely used to describe the orientation of various elements in the FIGURES. It should be noted that the orientation of various elements may differ according to other exemplary embodiments, and that such variations are intended to be encompassed by the present disclosure.

It is important to note that the construction and arrangement of the barrier device 10 as shown in the various exemplary embodiments is illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, manufacturing processes, etc.) without materially departing from the novel teachings and advantages of the subject matter described herein. For example, elements shown as integrally formed may be constructed of multiple parts or elements, the position of elements may be reversed or otherwise varied, and the nature or number of discrete elements or positions may be altered or varied. The order or sequence of any process or method steps may be varied or re-sequenced according to alternative embodiments. Other substitutions, modifications, changes and omissions may also be made in the design, operating conditions and arrangement of the various exemplary embodiments without departing from the scope of the present disclosure.

What is claimed is:

1. A barrier device, comprising:
a card including a top element and a bottom element coupled to a middle element;
wherein the middle element is sandwiched between the top and bottom elements;
wherein the middle element includes a hole disposed therethrough;
wherein the top element includes a hole disposed therethrough, the hole of the top element being smaller than the hole of the middle element;
wherein a flap is cut into the bottom element by cutting one or more lines therethrough;
wherein the hole of the top element is generally superimposed over the hole of the middle element when the top element is positioned over the middle element;
wherein the flap and the hole of the top element cooperatively define a one-way valve.

2. The barrier device of claim 1, wherein the top and bottom elements are polymeric.

3. The barrier device of claim 1, wherein a length of the card is approximately 3⅜ inches.

4. The barrier device of claim 1, wherein the middle element is opaque.

5. The barrier device of claim 1, wherein an upper surface of the top element defines an intake side of the barrier device, and wherein the intake side includes instructions for how to perform cardiopulmonary resuscitation displayed thereon.

6. The barrier device of claim 1, wherein the flap is configured to be coplanar with the bottom element when air is not flowing through the one-way valve.

7. The barrier device of claim 1, wherein the card remains generally flat when it is placed on the mouth of a person who is laying on his back.

8. A barrier device, comprising:
a card having a length dimension, a width dimension, and a height dimension, the length and width dimensions being substantially greater than the height dimension; and
a one-way valve;
wherein the one-way valve is coupled to a hole disposed within the card.

9. The barrier device of claim 8, wherein the length dimension of the card is approximately 3⅜ inches.

10. The barrier device of claim 8, wherein the card is comprised of a middle element coupled to a top element and a bottom element, and wherein the middle element is positioned between the top and bottom elements.

11. The barrier device of claim 10, wherein the top and bottom elements are polymeric.

12. The barrier device of claim 8, further comprising an intake side and an exhaust side;
wherein the one-way valve is configured such that air may be blown therethrough from the intake side to the exhaust side; and
wherein identification information is imprinted on either the intake side or the exhaust side.

13. The barrier device of claim 8, further comprising an intake side and an exhaust side;
wherein instructions for how to perform cardiopulmonary resuscitation are imprinted on either the intake side or the exhaust side.

14. The barrier device of claim 10, wherein the bottom and top elements are laminated onto the middle element.

15. The barrier device of claim 10, wherein the one-way valve is comprised of a hole disposed through the top element and a flap formed within the bottom element, and wherein the flap is configured to coplanar with the bottom element when air is not flowing through the one-way valve.

16. The barrier device of claim 15, wherein the flap is defined by an arc that is cut into the bottom element, and wherein an angle defined by a length of the arc is less than 180°.

17. A method for manufacturing a barrier device, the method comprising the following steps:
providing a planar middle element having a hole formed therein, a planar top element having a hole formed therein, and a planar bottom element having a flap formed therein; and
raising the flap from portions of the bottom element surrounding the flap, and coupling the middle, top, and bottom elements together; and
wherein the middle element is positioned between the top and bottom elements.

18. The method of claim 17, wherein the top and bottom elements are laminated onto the middle element.

19. The method of claim 17, wherein the flap is defined by an arc formed within the bottom element.

20. The method of claim 19, wherein an angle defined by a length of the arc is less than 180°.
